(19) United States
(12) Patent Application Publication
(10) Pub. No.: US 2008/0317852 A1
(43) Pub. Date: Dec. 25, 2008

(54) Pharmaceutical Combination

(76) Inventors: Amar Lulla, Maharashtra (IN); Geene Malhotra, Maharashtra (IN)

Correspondence Address: CONLEY ROSE, P.C., 5601 GRANITE PARKWAY, SUITE 750, PLANO, TX, 75024, US

(21) Appl. No.: 12/097,387

(22) PCT Filed: Dec. 14, 2006
(86) PCT No.: PCT/GB2006/004687

§ 371 Date: Aug. 19, 2008

(30) Foreign Application Priority Data
Dec. 14, 2005 (IN) 1566/MUM/2005
Nov. 13, 2006 (IN) 1878/MUM/2006

(51) Int. Cl. A61K 9/28; A61K 9/20; A61K 31/513; A61K 31/52; A61K 31/7072; A61K 31/675; A61K 31/538

(52) U.S. Cl. 424/465; 424/474; 424/464; 514/274; 514/263.4; 514/50; 514/81; 514/230.5

(57) ABSTRACT

A pharmaceutical formulation for the treatment of HIV is provided. The formulation is a combination of a nucleoside reverse transcriptase inhibitor and a nucleotide reverse transcriptase inhibitor in which the combination has an increased stability over prior art combination therapies. The invention also provides a pharmaceutical product containing the formulation.

### CROSS-REFERENCE TO RELATED APPLICATIONS

This application is a filing under 35 U.S.C. 371 of International Application No. PCT/GB2006/004687 filed Dec. 14, 2006, entitled “Pharmaceutical Combination,” claiming priority of Indian Patent Application Nos. 1566/MUM/2005 filed Dec. 14, 2005 and 1878/MUM/2006 filed Nov. 13, 2006, which applications are incorporated by reference herein in their entirety.

### FIELD OF THE INVENTION

The present invention relates to a pharmaceutical formulation for the treatment of human immunodeficiency virus (HIV) infection. In particular, the invention relates to a pharmaceutical formulation comprising a nucleoside reverse transcriptase inhibitor and a nucleotide reverse transcriptase inhibitor and to a pharmaceutical product containing the pharmaceutical formulation and a non-nucleoside reverse transcriptase inhibitor. The invention further relates to a process for preparing the pharmaceutical formulation and to its use in therapy.

### BACKGROUND OF THE INVENTION

Human Immunodeficiency virus (HIV) infection and related diseases are a major and global problem in today's world. HIV is the etiological agent of the complex disease that includes progressive suppression or destruction of the immune system known as Acquired Immune Deficiency Syndrome or AIDS and degeneration of the central and peripheral nervous system. HIV also predisposes subjects to fatal opportunistic infections.

HIV's genetic material is stored in the form of RNA. To allow incorporation of this genetic material into the host cell DNA, this viral RNA needs to be transformed into viral DNA. HIV is known as a retrovirus because it has this capability of copying RNA into DNA. Reverse transcriptase is a necessary enzyme for this reaction. To build its viral DNA, HIV uses nucleotides from the host cell's cytoplasm.

Nucleoside reverse transcriptase inhibitors (NRTIs) are nucleoside analogues that lack a 3′ hydroxyl group. After these nucleoside analogues have been phosphorylated (to the corresponding nucleotide), they can be incorporated into the growing DNA chain. Because of the missing 3′ hydroxyl group in these analogues, the newly made DNA strand is terminated early and polymerization by the reverse transcriptase is stopped. The activity of these NRTIs is not limited to HIV reverse transcriptase only, but can be used against other retroviruses also.

Tenofovir is a new nucleotide reverse transcriptase inhibitor recently approved in the United States for the treatment of HIV-1 infection in combination with other antiretroviral agents. Nucleotide analogues are very similar to nucleoside analogues but are pre-phosphorylated, and thus require less processing by the body. Tenofovir DF (disoproxil fumarate) is described in U.S. Pat. Nos. 5,935,946, 5,922,695, 5,977,089, 6,043,230 & 6,069,249 while PMPA or Tenofovir DF is described in U.S. Pat. Nos. 4,808,716, 5,733,788 & 6,057,305.

US2004/0224917 describes the combination of Tenofovir DF and Emtricitabine.

A common feature of retrovirus replication is the extensive post-translation processing of precursor polyproteins by a virally encoded protease to generate mature viral proteins required for virus assembly and function. Inhibition of this processing prevents the production of normally infectious virus. Literature reports that genetic inactivation of the HIV encoded protease results in the production of immature, non-infectious virus particles. These results indicate that inhibition of the HIV protease represents a viable method for the treatment of AIDS and the prevention or treatment of infection by HIV.

One substantial and persistent problem in the treatment of AIDS has been the ability of the HIV virus to develop resistance to the individual therapeutic agents employed to treat the disease.

Nowadays various combinations have been made available for this purpose and several attempts have been made to formulate combination regimens. One example is the combination of synthetic nucleoside analogues Lamivudine (150 mg) and Zidovudine (300 mg), which is commercially available as Combivir® of GlaxoSmithKline. Another such combination is of the nucleoside analogues Abacavir and Lamivudine, which is described in Glaxo's patent application no WO 03/101467.

Lamivudine (also known as 3TC) and its use in the treatment and prophylaxis of viral infections are described in U.S. Pat. No. 5,047,407. Lamivudine and its use against HIV are described in WO 91/17159 and EP 0382526. Crystalline forms of lamivudine are described in WO 92/21676.

Combinations of lamivudine with other nucleoside reverse transcriptase inhibitors, in particular zidovudine AZT, are described in WO 92/20344, WO 98/18477, and WO/9955372.

Various non-nucleoside reverse transcriptase inhibitors (NNRTIs), are known, such as delavirdine, capravirine, Efavirenz and nevirapine. NNRTIs are common components of therapy for antiretroviral-naive HIV-infected patients, and provide synergistic activity with nucleoside reverse transcriptase inhibitors (NRTIs).

Efavirenz is chemically known as (S)-6-chloro-4-(cyclopropylethynyl)-1,4-dihydro-4-(trifluoromethyl)-2H-3,1-benzoxazin-2-one. Efavirenz is a HIV-1 specific, non nucleoside, reverse transcriptase inhibitor. Efavirenz is useful for the treatment of HIV and has been reported to inhibit reproduction of HIV in the body.

Efavirenz is commercially available from Bristol-Myers Squibb Co, under the name SUSTIVA®, for treatment of HIV, and is described, for example, in U.S. Pat. Nos. 5,519,021, 5,663,1699, 5,811,423 and 6,238,695.

Nevirapine, chemically, 11-Cyclopropyl-5,11-dihydro-4-methyl-6H-dipyrido[3,2-b: 2′, 3′-e][1,4]diazepin-6-one is a non-nucleoside reverse transcriptase inhibitor. The therapeutic uses of nevirapine and related compounds and their preparations are described in U.S. Pat. No. 5,366,972. Nevirapine is commercially available as 200 mg tablet and 50 mg/5 mL in 240 mL oral suspension. It is sold under the name VIRAMUNE®.

Combination therapy reduces the daily dosages to be taken by patients and simplifies dosing schedule thereby increases patient compliance. Combination therapy also increases the drug efficacy. Use of combination therapy can yield an equivalent antiviral effect with reduced toxicity.

In spite of the existence of such combinations, there still remains a need to develop a combination for acute therapy and for resistant HIV viruses. It is thus an object of the present invention to provide a pharmaceutical composition which, inter alia, assists in treating the human immunodeficiency virus (HIV), and optionally related disorders resulting in AIDS.

### SUMMARY OF THE INVENTION

The present invention provides an effective combination which solves or alleviates the problems of the prior art. In particular, the present invention provides a pharmaceutical formulation in a single unit dosage form which has increased patient compliance and improved stability.

### DETAILED DESCRIPTION

In a first aspect, the invention provides a pharmaceutical formulation in a single unit dosage form, wherein the dosage form comprises:
(a) a nucleoside reverse transcriptase inhibitor or physiologically functional derivative thereof, and
(b) a nucleotide reverse transcriptase inhibitor or physiologically functional derivative thereof,
   wherein the nucleoside reverse transcriptase inhibitor or physiologically functional derivative thereof is provided in a different region of the dosage form to the nucleotide reverse transcriptase inhibitor or physiologically functional derivative thereof.

In a second aspect, there is provided a pharmaceutical product comprising a pharmaceutical formulation according to the invention, and further comprising a non-nucleoside reverse transcriptase inhibitor.

In a third aspect, there is provided a pharmaceutical product comprising:

i) lamivudine,

ii) a nucleotide reverse transcriptase inhibitor, and

iii) a non-nucleoside reverse transcriptase inhibitor.

In another aspect there is provided the use of a nucleoside reverse transcriptase inhibitor or physiologically functional derivative thereof and a nucleotide reverse transcriptase inhibitor or physiologically functional derivative thereof in the manufacture of a unitary dosage form pharmaceutical for the treatment or prevention of symptoms or effects of an HIV infection in an infected individual, wherein the nucleoside reverse transcriptase inhibitor or physiologically functional derivative thereof is provided in a different region of the dosage form to the nucleotide reverse transcriptase inhibitor or physiologically functional derivative thereof.

In another aspect, there is provided the use of a pharmaceutical formulation according to the invention, in the manufacture of a medicament for the treatment or prevention of symptoms or effects of an HIV infection in an infected individual.

The present invention further provides pharmaceutical compositions for simultaneous, separate or sequential use in the treatment or prevention of viral infections. In particular, the invention provides a pharmaceutical product comprising a pharmaceutical formulation according to invention and a non-nucleoside reverse transcriptase inhibitor for simultaneous, separate on sequential use.

The present invention thus provides an efficacious and long lasting therapy for AIDS which lowers HIV levels in patients to undetectable level and raises CD4 cell counts for prolonged periods without the development of resistance.

The combination therapies of the invention are a step ahead in the art for enhancing the effectiveness in treating AIDS and to preclude the development of resistance to individual therapeutic agents.

In the first aspect of the invention, the nucleoside reverse transcriptase inhibitor is preferably chosen from lamivudine, abacavir, emtricitabine, zidovudine, stavudine or physiologically functional derivatives thereof. Preferably lamivudine is used. The nucleotide reverse transcriptase inhibitor is preferably chosen from tenofovir DF or adefovir, and is preferably tenofovir DF.

In the second aspect of the invention, the non-nucleoside reverse transcriptase inhibitor is preferably chosen from efavirenz, nevirapine, delavirdine, or physiologically functional derivatives thereof. Preferably the non-nucleoside reverse transcriptase inhibitor is efavirenz.

In the third aspect of the invention, the nucleotide reverse transcriptase inhibitor is preferably chosen from tenofovir DF or adefovir or physiologically functional derivatives thereof. Preferably tenofovir DR is used. The non-nucleoside reverse transcriptase inhibitor is preferably chosen from efavirenz, nevirapine, delavirdine or physiologically function derivatives thereof. Preferably the non-nucleoride reverse transcription inhibitor is efavirenz.

The composition may be provided as an oral dosage form.

The composition or product according to the invention may be useful in the treatment of viral infections, particularly retroviral infections, which may include human immunodeficiency virus (HIV) and related disorders resulting in AIDS.

The term “physiologically functional derivative” as used herein means a pharmaceutically active compound with equivalent or near equivalent physiological functionality to the named active when administered according to the present invention. As used herein, the term “physiologically functional derivative” includes any pharmaceutically acceptable salts, solvates, esters, prodrugs derivatives, enantiomers, or polymorphs of the nucleoside-, nucleotide- or non-nucleoside reverse transcriptase inhibitors.

A layered tablet (bilayered, trilayered, etc.) as used herein refers to a tablet in which two or more layers of active material have been compressed successively. Such tablets are also known as laminated tablets. Both or all layers of active material are exposed (although the tablet may be further coated). This differs from a core or coated core tablet, in which the core of a first active material is circumscribed, and thus concealed from the exterior of the tablet, by a coating layer of another active material.

Further, the invention provides a method for treating, reducing or inhibiting retroviral infections, in particular HIV infections in a mammal, which includes administering to a human, a safe and effective amount of a pharmaceutical formulation or product according to the invention.

Reference to word “treatment” as used herein extends to both the prophylaxis and the treatment of an established malady, infection or its symptoms.

HIV causes a variety of clinical conditions including acquired immunodeficiency syndrome (AIDS) and chronic neurological disorders. Multiple drug regimes dramatically improve the treatment of HIV infected patients.

Single drug treatment regimens typically require long term treatment increasing the evidence of unwanted side effects. Moreover, single drug therapies are particularly vulnerable to mutation in the HIV runs, leading to drug resistant variants of HIV.

The use of multiple drug therapies may reduce the development of drug resistant strains of HIV because one drug will usually cancel out mutations against other drugs. Multiple drug therapies even inhibit replication of HIV viruses for a period of time sufficient to eliminate HIV from the body. However, not all combinations of drugs are equally efficacious, and some combinations can be ineffective or have undesirable side effects. Moreover, certain drug combinations can result in undesirably poor stability.

The effective multiple drug treatments for HIV often require strict compliance with a complex treatment regimen that can require the administration of many different drugs per day, administered at a precisely timed intervals with careful attention to diet. Patient non-compliance is a well-known problem accompanying such complex treatment regimens in the treatment of HIV because such non-compliance may lead to the emergence of multiple drug resistant strains of HIV and also abandonment of treatment in the middle of the therapy.

The combination therapy in accordance with the invention thus provides a method to enhance the effectiveness in treating AIDS and to prevent the development of resistance to the individual therapeutic agents.

It will be appreciated, therefore, that the pharmaceutical combinations of the present invention, and in particular the preferred combination of lamivudine, tenofovir DF or a physiologically functional derivation thereof, and efavirenz or a physiologically functional derivative thereof, provide significant advantages over the prior art. The combination may conveniently be presented as individual pharmaceutical formulations in unitary dosage form. In this respect the present invention provides a pharmaceutical kit or product comprising (i) a first pharmaceutical formulation comprising lamivudine, together with one or more pharmaceutically acceptable carriers or excipients, and tenofovir DF or a physiologically functional derivative thereof, together with one or more pharmaceutically acceptable carriers or excipients; and (ii) a second pharmaceutical formulation comprising efavirenz or a physiologically acceptable salt thereof, together with one or more pharmaceutically acceptable carriers or excipients; for separate or sequential use in the treatment or prevention of viral infections.

The pharmaceutical kit or product may be provided in a patient pack, optionally comprising an information insert containing directions on the use of the kit/product.

Lamivudine (also known as 3TC) is a synthetic nucleoside analogue, chemically known as (2R,cis)-4-amino-1-(2-hydroxymethyl-1,3-oxathiolan-5-yl)-(1H)-pyrimidin-2-one (Epivir®). Lamivudine has proven antiviral activity against HIV and other viruses such as HBV.

Intracellularly, lamivudine is phosphorylated to its active 5′-triphosphate metabolite, lamivudine triphosphate (L-TP). The principal mode of action of L-TP is the inhibition of HIV-1 reverse transcriptase (RT) via DNA chain termination after incorporation of the nucleoside analogue into viral DNA. L-TP is a weak inhibitor of mammalian DNA polymerases (alpha) and (beta), and mitochondrial DNA polymerase (gamma).

Lamivudine has also been referred to as (−)-1-[(2R,5S) 2-(Hydroxymethyl)-1,3-oxathiolan-5-yl]cystosine, (Hydroxymethyl)-1,3-oxathiolan-5-yl]cystosine and it has proven antiviral activity against human immunodeficiency virus (HIV) and other viruses such as hepatitis B. Lamivudine is commercially available from Glaxo Wellcome Inc under trade name EPIVIR.

Tenofovir disoproxil fumarate is also known as PMPA. Tenofovir DF (a prodrug of tenofovir) is a fumaric acid salt of bis-isopropoxycarbonyloxymethyl ester derivative of tenofovir. Tenofovir disoproxil fumarate is 9-[(R)-2-[[bis[[(isopropoxycarbonyl)oxy]methoxy]phosphinyl]methoxy]propyl]adenine fumarate (1:1). Tenofovir disoproxil fumarate requires initial diester hydrolysis for conversion to tenofovir and subsequent phosphorylations by cellular enzymes to form tenofovir diphosphate. Tenofovir diphosphate inhibits the activity of HIV reverse transcriptase by competing with the natural substrate deoxyadenosine 5′-triphosphate and, after incorporation into DNA, by DNA chain termination. Tenofovir diphosphate is a weak inhibitor of mammalian DNA polymerases alpha & beta and of mitochondrial DNA polymerase.

Tenofovir disoproxil fumarate is an analog of adefovir and is classified as a nucleotide reverse transcriptase inhibitor (NtRTI). Tenofovir DF is a competitive inhibitor of other naturally occurring nucleotides, and its ultimate biological activity is viral DNA chain termination. Tenofovir DF is a novel nucleotide analog with antiviral activity against both HIV and HBV. The mechanism of tenofovir DF is similar to that of nucleoside analogs, which interferes with reverse transcriptase and prevents translation of viral genetic material into viral DNA. Unlike the nucleoside analogs, the nucleotide reverse transcriptase inhibitors are chemically pre-activated with the presence of phosphate group. Since the phosphorylation step is not necessary, nucleotide analogs can incorporate into viral DNA chain more rapidly than nucleoside analogs. More importantly, this will bypass a viral mechanism of nucleoside resistance. Tenofovir DF is commercially available from Gilead Science Inc. under the trade name VIREAD®.

The chemical stability of the active ingredients in a pharmaceutical formulation is of significant concern, as is the appearance of the formulation and how it changes over time. The inventors of the present invention have surprisingly found that Lamivudine and Tenofovir DF, when intimately mixed to form a (single layered) tablet showed undesirable properties in stability testing. The appearance of tablets changed to brown colour at Controlled Room Temperature (25°) and even at Accelerated temperature (40° C.). However, the inventors have surprisingly found that such a change in appearance is not found in case of a bilayered tablet.

Product: Lamivudine & Tenofovir Disoproxil Fumarate Tablets

| CONDITION | | 25° C./60% RH | 40° C./75% RH |
|---|---|---|---|
| PARAMETERS | INITIAL | 1 M | 1 M |
| Appearance | Single | Brown | Brown |
| | layered | colouration | colouration |
| | tablets | | with gas |
| | | | formation. |
| Appearance | Bilayered | No change | No change |
| | tablets | in the | in the |
| | | colour | colour |

By means of a pharmaceutical formulation according to the present invention comprising a bilayered tablet comprising an NRTI and an NtRTI, treatment regimens for HIV and other viruses can be simplified with the goal of enhancing patient compliance by providing a simplified dosage therapy.

It is also possible to combine any two of the NRTIs and NtRTIs employed in the present invention in a unitary dosage form for separate or sequential administration with a separate dosage form comprising an NNRTI. In a preferred embodiment, a typical unitary dosage may contain lamivudine and tenofovir DF, or physiologically functional derivatives thereof, and a further unitary dosage may contain efavirenz, or a physiologically functional derivative thereof. In this respect, the present invention provides a pharmaceutical product comprising (i) a first pharmaceutical formulation comprising lamivudine and tenofovir DF or physiologically functional derivatives thereof, optionally in the form of a bilayered tablet, together with one or more pharmaceutically acceptable carriers or excipients; and (ii) a second pharmaceutical formulation comprising efavirenz, or a physiologically functional derivative thereof together with one or more pharmaceutically acceptable carriers or excipients, for separate or sequential use in the treatment or prevention of viral infections, in particular retroviral infections, and especially the symptoms or effects of HIV infection, in an infected animal.

The pharmaceutical product and formulation of the present invention employ a combination of safe and therapeutically effective amount of at least two therapeutically active agents and preferably three therapeutically active agents, such as a safe and therapeutically effective amount of 2′,3′-dideoxy-3′-thiacytidine (lamivudine) or its physiologically functional derivatives, a safe and therapeutically effective amount of tenofovir DF, (R)-9-(2-phosphonylmethoxypropyl) adenine or its physiologically funcational derivatives, optionally with a safe and therapeutically effective amount of efavirenz or its physiologically functional derivatives, along with safe and effective amounts of pharmaceutically acceptable excipients to maintain the homogeneity of the dosage forms.

The composition may be provided in the form of tablets or capsules.

The pharmaceutical product of the present invention conveniently allows administration of a pharmaceutical combination a separate or subsequent dosage containing three active compounds in oral dosage forms containing specific dosage ranges for each compound. Preferably a unity dosage form is provided comprising an NRTI and an NtRTI. Preferably an additional dosage form is provided comprising an NNRTI, for separate or sequential administration.

Lamivudine may be present preferably in a range of 5-600 mg and most preferably 300 mg per unit dosage form.

Tenofovir DF may be present preferably in a range of 75-600 mg and preferably 300 mg per unit dosage form.

Efavirenz may be present preferably in a range of 50-600 mg and preferably 600 mg per unit dosage form.

The formulation and product of the present invention may further comprise pharmaceutical excipients to impact beneficial characteristics to the dosage form. Typical excipients include, diluents or bulking agents, fillers, disintegrants, binders, lubricants, coating materials, wetting agents and the like.

Where present, a diluent or bulking agent can be selected to provide an increase in tablet size. The skilled person can utilize known methods to select a bulking agent, which provides hardness, friability and disintegration time required for pharmaceutical advantage. Suitable diluents included microcrystalline cellulose, lactose and the like. The diluent is preferably present in an amount of from 5% to 50% by weight of the formulation.

Fillers suitable for use with the present invention may comprise one or more of sugars, sugar alcohols, starches, and inert materials, such as kaolin and the like, that add to the bulk of the formulation.

Disintegrating agents suitable for use with the present invention may comprise one or more of celluloses and their derivatives, alginates, agar-agar, certain complex silicilates, starches, modified starches and their derivatives, polyvinylpyrolidones and the like. Preferred disintegrants include sodium starch glycollate and/or croscarmellose sodium. The disintegrant is preferably present in an amount of from 0.5% to 30% by weight of the formulation.

Binders may comprise one or more of but not limited to, natural and synthetic gums, celluloses, starches, gelatins and povidones and the like. Preferably, the binder comprises starch, Maltodextrins, HPMC, HPC and/or povidone. The binder is preferably present in an amount of from 1% to 50% by weight of the formulation.

Lubricants suitable for use with the present invention may comprise one or more of talc, magnesium stearate, starch, dextrin, sodium stearyl fumarate, hydrogenated vegetable oils, polyethylene glycols and their derivatives, sodium lauryl sulphate and the like. Preferably, the lubricants comprise one or more of magnesium stearate, zinc stearate, calcium stearate and sodium stearyl fumarate. More preferably the lubricant is magnesium stearate. The lubricant is preferably present in an amount of from 0.25% to 3% by weight of the formulation.

The tablets may be coated for the purpose of providing protection from moisture. The coating material can be selected from one or more of celluloses and their derivatives, polyethylene glycols and their derivatives, fatty acids such as stearic acid and their derivatives, and waxes, among other suitable coating materials well known in the art.

The person skilled in the art may include wetting agent such as polysorbate 80, SLS, sucrose esters, polyethylene glycols, lutrols, cremophor and the like. Where present, the wetting agent is preferably present in an amount of from 0.1% to 5%, by weight of the formulation.

In a preferred embodiment, the formulation is in the form of a bilayered tablet. In another preferred embodiment the product is in the form of a bilayered tablet and a subsequent unitary tablet formulation.

In these embodiments, the first layer of said bilayered tablet preferably contains about 5 to 55% wt. lamivudine or a physiologically functional derivative thereof, about 1 to 50% wt. diluents, about 1 to 50% wt. binders, about 1 to 30% wt. disintegrant and about 0.25 to 3.0% wt. of a lubricant.

More preferably, the first layer of tablet containing lamivudine or a physiologically functional derivative thereof in the formulation or product according to the present invention comprises 5 to 55% wt. of lamivudine, about 10 to 50% wt. microcrystalline cellulose, about 2 to 30% wt. Sodium starch glycolate, about 1 to 10% wt. of starch and about 0.25 to 2.5% wt. of a magnesium stearate.

In these embodiments the second layer of said bilayered tablet preferably contains about 10 to 85% wt. of tenofovir DF or a physiologically functional derivative thereof, about 1 to 50% wt. diluent, about 1 to 50% wt. binder, about 0.5 to 30% wt. disintegrant and about 0.25 to 3% wt. of a lubricant.

More preferably, the second layer of tablet comprises about 35 to 85% wt. of tenofovir DF, about 5 to 50% wt. lactose or microcrystalline cellulose, about 1 to 10% wt. starch, about 1 to 20% wt. sodium starch glycollate and about 0.2 to 2% wt. of magnesium stearate.

In the preferred pharmaceutical product of the present invention, a unitary tablet dose is preferably provided containing at least one non-nucleoside reverse transcriptase inhibitor or a physiologically functional derivative thereof. In the preferred product according to the present invention said unitary dosage form comprises about 10 to 50% wt. of efavirenz or a physiologically functional derivative thereof, about 1 to 50% wt. diluent, about 1 to 50% wt. binder, about 0.5 to 30% wt. disintegrant and about 0.2 to 3% wt. of a lubricant.

Different techniques known in the art may be employed to formulate granules. In a preferred embodiment of the present invention, a method of preparing a pharmaceutical composition for a first layer of said bilayered tablet preferably includes the steps of blending a diluent and disintegrant with lamivudine; granulating it further with water and suitable binder into granules; drying the resulting granules; sizing and lubricating the granules.

A preferred method of preparing a pharmaceutical composition for a second layer of said bilayered tablet preferably includes the steps of blending a diluent with Tenofovir DF; granulating it further with water and suitable binder into granules; drying the resulting granules and sizing; again blending with suitable colour and disintegrant; lubricating the granules.

Lubricated granules of both the layers may then be compressed together using suitable compression machine.

Both the layers may optionally contain colouring agents.

A preferred method of manufacturing the unitary tablet preferably includes the steps of blending a diluent with efavirenz; granulating it further with water and suitable binder into granules; drying the resulting granules and sizing; blending with suitable colour (if desired) and disintegrant; lubricating the granules.

The present invention may be formulated as a multi-layered tablet formulation, preferably bilayered which can typically be administered to patients and permits or achieves delivery of pharmaceutically active agents effective for the prevention or treatment of infection by HIV and in prevention or treatment of the resulting acquired immunodeficiency syndrome (AIDS).

The present invention formulated as a bilayered tablet may be further modified to act as a dispersible tablet comprising suitable excipients known to the person skilled in the art.

Each part of the tablet of the present invention can be prepared by wet granulation or direct compression or dry granulation. In view of the relatively high dosage of lamivudine it is preferred to use wet granulation techniques.

The formulation of the present invention may also be formulated as a trilayered tablets. In this embodiment, a placebo layer is used as an intermediate layer between the NNRT and NtRTI layers. Preferably the placebo layer comprises pharmaceutical excipients but does not comprise any active ingredient. One suitable placebo comprises silica gel. This may further increase the stability of a tablet containing, for example, lamivudine and tenofovir DF in distinct layers.

In a preferred embodiment of the present invention, a pharmaceutical product comprises a bilayered system with a subsequent unitary tablet formulation including the pharmaceutically active agents effective for the treatment of HIV. More particularly, a product according to the present invention preferably comprises bilayered formulation comprising a first layer comprising at least one nucleoside reverse transcriptase inhibitor, optionally further comprising pharmaceutical excipients, and a second layer comprising at least nucleotide reverse transcriptase inhibitor, optionally further comprising pharmaceutical excipients; and a formulation comprising a non-nucleoside reverse transcriptase inhibitor wherein the bilayered formulation form a pharmaceutically stable preparation together.

### EXAMPLES

The invention will now be described further with reference to the following examples which are specific embodiments only and are not limiting on the scope of the invention.

### Example 1

| | |
|---|---|
| INGREDIENTS | QTY (mg/tab) |

(A) Lamivudine & Tenofovir DF (disoproxil fumarate) tablet
Layer I

| | |
|---|---|
| Lamivudine | 150.00 |
| Microcrystalline cellulose | 99.50 |
| Sodium starch glycollate | 40.00 |
| Magnesium stearate | 4.00 |
| Color | 5.00 |

Layer II

| | |
|---|---|
| Tenofovir DF | 300.00 |
| Lactose | 170.00 |
| Microcrystalline cellulose | 95.00 |
| Starch | 30.00 |
| Polysorbate 80 | 6.50 |
| Croscarmellose sodium | 25.00 |
| Magnesium stearate | 6.50 |
| Colour | 0.50 |

(B) Efavirenz tablet
Intra-granular ingredients:

| | |
|---|---|
| Efavirenz | 600.00 |
| Lactose | 495.00 |
| Sodium starch glycolate | 50.00 |

Binder:

| | |
|---|---|
| Povidone (PVP K-30) | 40.00 |
| Purified water | q.s. |

Extra granular ingredients:

| | |
|---|---|
| Sodium starch glycolate | 50.00 |
| Pregelatinised starch | 50.00 |
| Magnesium stearate | 15.00 |
| Colour | 15.00 |
| Purified water | q.s. |

### Process for Preparation of Bilayer Tablet

The preparation of layer-I includes the steps of blending of diluent, disintegrant and optionally suitable colour with Lamivudine and then lubricating the blend.

The preparation of layer-II includes the steps of blending of diluent with Tenofovir DF; further granulating it with water and suitable binder to obtain granules; drying the resulting granules and sizing the granules; again blending the said granules with suitable colour and disintegrant; and lubricating the granules.

Lubricated granules of both the layers then compressed together using suitable compression machine.

Preparation Efavirenz Tablet Includes the Step of Blending of Diluent with Efavirenz; further granulating it with water and suitable binder to obtain granules; drying the resulting granules and sizing the granules; again blending the said granules with suitable colour and disintegrant; and lubricating the granules.

### Example 2

| | |
|---|---|
| INGREDIENTS | QTY (mg /tab) |

Lamivudine Layer

| | |
|---|---|
| Lamivudine | 300.00 |
| Microcrystalline cellulose | 98.40 |
| Sodium starch glycollate | 50.00 |
| Colour | 0.60 |
| Starch (binder) | 15.00 |
| Purified water | q.s. |
| Magnesium stearate | 6.00 |

Tenofovir Layer

| | |
|---|---|
| Tenofovir disoproxil fumarate equivalent | 300.00 |
| to Tenofovir disoproxil | |
| Lactose | 159.50 |
| Croscarmellose sodium | 40.00 |
| Starch | 45.00 |
| Polysorbate 80 | 3.00 |
| Water | q.s. |
| Microcrystalline cellulose | 100.00 |
| Magnesium stearate | 12.50 |

Process of manufacture for Tenofovir layer—A premix of tenofovir and lactose is prepared. This is drymixed with croscarmellose sodium and starch. A binder solution of starch and polysorbate 80 in purified water is prepared. The drymix is granulated using the binder solution, wet granules are dried and sized and lubricated.

For the lamivudine layer—Drymix of lamivudine, microcrystalline cellulose, sodium starch glycollate and colour is prepared. This is granulated with a binder solution (starch paste). The granules are sized, dried and lubricated.

The tablets are compressed and coated using a colour redimix solution.

It will be appreciated that the invention may be modified.

1. A pharmaceutical formulation in a single unit dosage form, wherein the dosage form comprises:
(a) a nucleoside reverse transcriptase inhibitor or physiologically functional derivative thereof, and
(b) a nucleotide reverse transcriptase inhibitor or physiologically functional derivative thereof,
wherein the formulation is in the form of a multi-layered tablet or a coated core tablet, and wherein the nucleoside reverse transcriptase inhibitor or physiologically functional derivative thereof is provided in a different region of the dosage form to the nucleotide reverse transcriptase inhibitor or physiologically functional derivative thereof.

2.-45. (canceled)

46. The pharmaceutical formulation according to claim 1, wherein the nucleoside reverse transcriptase inhibitor or physiologically functional derivative thereof is provided in a first region of the dosage form, and the nucleotide reverse transcriptase inhibitor or physiologically functional derivative thereof provided in a second region of the dosage form, wherein the first region is free, or substantially free of the nucleotide reverse transcriptase inhibitor, and the second region is free or substantially free of the nucleoside reverse transcriptase inhibitor.

47. The pharmaceutical formulation according to claim 1, which is in the form of a multi-layered tablet, a first layer of said tablet comprising said nucleoside reverse transcriptase inhibitor or physiologically functional derivative thereof and a second layer of said tablet comprising said nucleotide reverse transcriptase inhibitor or physicologically functional derivative thereof.

48. The pharmaceutical formulation according to claim 47, wherein the multi-layered tablet is a bilayered tablet or a trilayered tablet.

49. The pharmaceutical formulation according to claim 48, wherein the multi-layered tablet is a bilayered tablet.

50. The pharmaceutical formulation according to claim 48, wherein the multi-layered tablet is a trilayered tablet, and wherein a third layer of said tablet is free or substantially free of the nucleoside reverse transcriptase inhibitor and the nucleotide reverse transcriptase inhibitor.

51. The pharmaceutical formulation according to claim 50, wherein the third layer comprises no or substantially no pharmaceutically active agent.

52. The pharmaceutical formulation according to claim 50, wherein the third layer is intermediate said first and second layers.

53. The pharmaceutical formulation according to claim 46, wherein the coated tablet is formed of a core comprising one of said first or second regions, and a coating layer comprising the other of said first or second regions.

54. The pharmaceutical formulation according to claim 53, wherein the coated tablet comprises an intermediate layer interposed between to said core and coating layer.

55. The pharmaceutical formulation according to claim 54, wherein the intermediate layer of said tablet comprises none or substantially none of the nucleoside reverse transcriptase inhibitor and the nucleotide reverse transcriptase inhibitor.

56. The pharmaceutical formulation according to claim 55, wherein the intermediate layer comprises a placebo or a non-pharmaceutically active agent.

57. The pharmaceutical formulation according to claim 1, wherein the nucleoside reverse transcriptase inhibitor is lamivudine, abacavir, emitricitabine, zidovudine or stavudine, or a physiologically functional derivative thereof.

58. The pharmaceutical formulation according to claim 57, wherein the nucleoside reverse transcriptase inhibitor is lamivudine or a physiologically functional derivative thereof.

59. The pharmaceutical formulation according to claim 1, wherein the nucleotide reverse transcriptase inhibitor is tenofovir DF or adefovir, or a physiologically functional derivative thereof.

60. The pharmaceutical formulation according to claim 59, wherein the nucleotide reverse transcriptase inhibitor is tenofovir DF.

61. The pharmaceutical formulation according to claim 1, wherein the nucleoside reverse transcriptase inhibitor is lamivudine and the nucleotide reverse transcriptase inhibitor is tenofovir DF.

62. The pharmaceutical formulation according to claim 1, comprising from 50 mg to 600 mg of said nucleoside reverse transcriptase inhibitor, per unit dosage form.

63. The pharmaceutical formulation according to claim 1, comprising from 150 mg to 450 mg of said nucleoside reverse transcriptase inhibitor, per unit dosage form.

64. The pharmaceutical formulation according to claim 1, comprising approximately 300 mg of said nucleoside reverse transcriptase inhibitor, per unit dosage form.

65. The pharmaceutical formulation according to claim 1, comprising from 75 mg to 600 mg of said nucleotide reverse transcriptase inhibitor, per unit dosage form.

66. The pharmaceutical formulation according to claim 1, comprising from 150 mg to 450 mg of said nucleotide reverse transcriptase inhibitor, per unit dosage form.

67. The pharmaceutical formulation according to claim 1, comprising approximately 300 mg of said nucleotide reverse transcriptase inhibitor.

68. The pharmaceutical formulation according to claim 47, wherein the first and/or second layer further comprises at least one pharmaceutically acceptable excipient.

69. The pharmaceutical formulation according to claim 68, wherein the pharmaceutically acceptable excipient comprises at least one of diluent, filler, bulking agent distintegrant, binder or lubricant.

70. The pharmaceutical formulation according to claim 1, which is in the form of a tablet, optionally a bilayered tablet, wherein said tablet is coated.

71. The pharmaceutical formulation according to of claim 47, wherein the first layer comprises 5 to 55% wt. nucleoside reverse transcriptase inhibitor, 0.5 to 30% wt. diluent, 1 to 30% wt. disintegrant, 1 to 50% wt. binder and 0.25 to 3.0% wt. lubricant.

72. The pharmaceutical formulation according to claim 47, wherein the first layer comprises 5 to 55% wt. Lamivudine or a physiologically functional derivative thereof, 0.5 to 30% wt. diluent, 1 to 30% wt. disintegrant, 1 to 50% wt. binder and 0.25 to 3.0% wt. lubricant.

73. The pharmaceutical formulation according to claim 47, wherein the second layer comprises 10 to 85% wt. nucleotide reverse transcriptase inhibitor, 1 to 50% wt. diluent, 1 to 50% wt. binder, 0.5 to 30% wt. disintegrant and 0.25 to 3% wt. lubricant.

74. The pharmaceutical formulation according to claim 47, wherein the second layer comprises 35 to 85% wt. 5 to 50% wt. diluent, 1 to 10% wt. binder, and 0.2 to 2% wt. lubricant.

75. The pharmaceutical formulation according to claim 71, wherein the nucleotide reverse transcriptase inhibitor is tenofovir DF.

76. A pharmaceutical product comprising the pharmaceutical formulation according to claim 1, and further comprising a non-nuclcoside reverse transcriptase inhibitor.

77. The pharmaceutical product according to claim 76, wherein the non-nucleoside reverse transcriptase inhibitor is efavirenz, nevirapine or delavirdine, or a physiologically functional derivative thereof.

78. The pharmaceutical product according to claim 76, wherein the non-nucleoside reverse transcriptase inhibitor is efavirenz.

79. A pharmaceutical product comprising:
i) Lamivudine,
ii) a nucleotide reverse transcriptase inhibitor, and
iii) a non-nucleoside reverse transcriptase inhibitor
wherein the product is in the form of a multi-layered tablet or a coated core tablet, and wherein the nucleoside reverse transcriptase inhibitor is provided in a different region of the dosage form to the lamivudine.

80. The pharmaceutical product according to claim 79, wherein the nucleotide reverse transcriptase inhibitor is tenofovir DF.

81. The pharmaceutical product according to claim 80, wherein the non-nucleoside reverse transcriptase inhibitor is efavirenz.

82. The pharmaceutical product according to claim 81, wherein the lamivudine and the nucleotide reverse transcriptase inhibitor are formulated as a pharmaceutical formulation according to any of claims 1 to 31.

83. The pharmaceutical product according to claim 82, wherein the non-nucleoside reverse transcriptase inhibitor is formulated as a first dosage form, and the nucleoside reverse transcriptase inhibitor and the nucleoside reverse transcriptase inhibitor are formulated as a second, separate, unitary dosage form.

84. The pharmaceutical product according to claim 83, wherein the separate dosage form comprises 10 to 50% wt. of said non-nucleoside reverse transcriptase inhibitor, 1 to 50% wt. diluent, 1 to 50% wt. binder, 0.5 to 30% wt. disintegrant and 0.2 to 3% wt. lubricant.

85. The pharmaceutical product according to claim 84, wherein the separate dosage form is in the form of a tablet, powder or tablets in capsules, tablet in tablet, pellets, granules, oral powder, solution or suspension.
